(19) United States
(12) Patent Application Publication
(10) Pub. No.: US 2004/0028678 A1
(43) Pub. Date: Feb. 12, 2004

(54) Use of activated layered silicates for the adsorption of mycotoxins

(76) Inventors: Norbert Schall, Forstern (DE); Hubert Simmler-Hubenthal, Moosburg (DE)

Correspondence Address: Scott R Cox, Lynch Cox Gilman & Mahan, Suite 2200, 400 West Market Street, Louisville, KY, 40202, US

(21) Appl. No.: 10/416,082

(22) PCT Filed: Oct. 10, 2001
(86) PCT No.: PCT/EP01/11713

(30) Foreign Application Priority Data
Nov. 15, 2000 (DE) 100 56 634.0

(51) Int. Cl. A61K 39/00

(52) U.S. Cl. 424/140.1

(57) ABSTRACT

The invention concerns the use of an acid-activated layer silicate for adsorption of mycotoxins, in which layer silicate is activated with less than 10 wt %, referred to absolutely dry layer silicate of an acid at a temperature below 80° C. The invention also concerns a feed preparation and a method for feed treatment in which this mycotoxin adsorbent is used.

### DESCRIPTION

The invention concerns the use of layer silicates activated with acid for adsorption of toxins, especially mycotoxins in feeds, a method for improvement of usability of mycotoxin-contaminated feed, as well as a feed preparation containing the mycotoxin adsorbent.

The term mycotoxins includes a group of toxic substances that are formed by different naturally occurring fungi. About 300 to 400 mycotoxins are now known. Cereals and grains are considered the natural environment for these fungi. Whereas some types of fungi already develop on the still ripening grain in the spike, other species mostly attack grain-feed supplies being stored, when a certain minimum humidity and ambient temperature are present.

All so-called mycotoxins have a harmful effect on health primarily on agricultural animals fed with contaminated grains, but secondarily on humans via the food chain.

The following mycotoxins are significant in animal and also human nutrition worldwide with different regional expression: aflatoxin, ochratoxin, fumonisin, zearalenone, deoxynivalenol, T2 toxin and ergotamine. For a further discussion of these and additional mycotoxins, WO 00/41806 of the same applicant and the sources mentioned there can be referred to.

Several different toxins could be determined in different feeds by the development of more sensitive analysis methods and these toxins have been recognized as the causal agents of health problems in man and animals. A number of studies were able to demonstrate that several toxins can occur simultaneously in feeds. This simultaneous occurrence can significantly influence the toxicity of the mycotoxins. In addition to acute damage to agricultural animals that receive mycotoxin-contaminated feed, health damage in humans is also being discussed in the literature, developing from continuous adsorption of foods weakly contaminated with mycotoxins.

In a recent study of suspicious feed samples, aflatoxin, deoxynivalenol or fumonisin were found in more than 70% of the investigated samples (cf. “Understanding and coping with effects of mycotoxins in life dog feed and forage”, North Carolina Cooperative Extension Service, North Carolina State University; http:/www.ces.ncsu.edu/drought/dro-29.html).

In many cases the economic effects with reference to reduced productivity of the animals, increase the occurrence of diseases by immune suppression, damage to vital organs and an adverse effect on reproductivity are greater than the effects caused by death of the animals from mycotoxin intoxication.

A group of aflatoxins because of their specific molecular structure is fixed with high specificity on some mineral adsorbents (like zeolite, bentonite, aluminum silicate and others (cf. A.-J. Ramos, J. Fink-Gremmels, E. Hernandez, “Prevention of Toxic Effects of Mycotoxins by Means of Nonnutritive Adsorbent Compounds,” J. of Food Protection, Vol. 59(6), 1996, pp. 631-641).

U.S. Pat. No. 5,149,549 describes and claims the use of bentonite as a mycotoxin- but especially aflatoxin-adsorbent for use in animal feeds.

However, binding of other aforementioned important mycotoxins on natural mineral adsorbents occurs with only very limited effectiveness. To improve the adsorbent capacity of mineral adsorbents for these non-aflatoxins, different types of surface modification have been proposed in natural silicates.

A dry particulate animal feed additive containing phyllosilicate particles coated with a sequestering agent, for example EDTA, is described in WO 91/13555.

S. L. Lemke, P. G. Grant and T. D. Phillips describe an organically modified (organophilic) montmorillonite clay that is capable of adsorbing zearalenone in “Adsorption of Zearalenone by Organophilic Montmorillonite Clay,” J. Agric. Food Chem. (1998), pp. 3789-3796.

An improved mycotoxin adsorption capacity by organically modified (organophilic) layer silicates is also disclosed and claimed by the same applicant in WO 00/41806.

A common feature of the organically modified (organophilic) adsorbents, however, is that they only bind a selection of specific toxins with high efficiency, or as other toxins, like fumonisin cannot be effectively bonded even with organophilic surface modification.

EP 0 721 741 A1 discloses and claims a method and composition for improvement of the nutritional value of mycotoxin-contaminated animal feeds. The acid-activated montmorillonite clay used there is produced by adding acid to a clay suspension, in which the most uniformly activated clay material possible is supposed to be achieved. About 10 to 35 wt % acid is then converted at temperatures of 80 to 100° C. over several hours with a layer of silicate.

By this type of acid modification, the adsorption performance for toxins that can be bonded in particular to acid surfaces (like fumonisin) does increase, but the capacity to bind other toxins is reduced. The aforementioned conventional acid activation is also a demanding and therefore costly process.

The task of the invention is therefore to prepare a mycotoxin adsorbent that avoids the drawbacks of the prior art and permits efficient adsorption of the broadest possible spectrum of different mycotoxins, especially efficient adsorption of those toxins that can primarily be bonded to acid surfaces (for example, fumonisin) without simultaneously reducing the binding capacity for other toxins.

This task is solved by the use of an acid-activated layer silicate according to claim 1.

It was surprisingly found that a particularly good mycotoxin adsorbent is obtained if, referred to the dry weight of layer silicate (layer silicate air dried), less than 10 wt % of a mineral acid, preferably less than 8 wt %, especially less than 6 wt % of an acid is used for activation of the layer silicate and an activation temperature below 80° C. is maintained.

It was also found that particularly good adsorbents are obtained if activation occurs by spraying of the starting material with the acid. This so-called dry activation process, in contrast to conventional acid activation, is characterized by conversion with acid in a suspension at high temperature owing to the fact that no liquid wastewater is formed. The same applies for kneading in of limited amounts of acid according to the invention. The dry activation process can also occur at room temperature according to a preferred variant.

According to a preferred variant, the layer silicate is activated with only about 0.5 to 8 wt %, especially 1 to 6 wt %, in particular about 1.5 to 4 wt % acid.

The acid effect time is dependent on the employed amount of acid and the activation temperature, but activation times of less than 2 hours, especially less than 1 hour, are generally sufficient.

Any phyllosilicate that can be activated with acid can be used as layer silicate according to the invention. A layer silicate from the smectite group, the serpentine-kaolin group, the talc-pyrophyllite group, the group of attapulgite/palygorskite, vermiculite, illite, sepiolite and/or the mica-like layer silicates are preferably used as layer silicate.

The layer silicates of the smectite group include the trioctahedral smectites, like saponite and hectorite, and the dioctahedral smectites, like montmorillonite, beidellite and nontronite. The serpentine-kaolin group includes, for example, chrysotile, antigorite, kaolinite and halloysite. Talc and pyrophyllite belong to the talc-pyrophyllite group. The trioctahedral and dioctahedral chlorites belong to the chlorite group.

Montmorillonite clays, like smectites, especially bentonites as well as attapulgite and halloysite, as well as their naturally occurring mixtures are particular preferred as layer silicates.

According to a particularly preferred variant, the layer silicate is a mixture of attapulgite and bentonite in which the attapulgite fraction with further preference lies between about 10 and about 90 wt %, especially about 20 to 60 wt %.

The layer silicate also preferably has a pore volume in the range from 0.1 to 0.5 cm³/g.

Among the mentioned layer silicates, one the has a pH value of less 9, preferably about 4.0 to 8.0 in an aqueous suspension is preferably used as starting material.

According to a particularly preferred variant, a layer silicate with an ion exchange capacity (IEC) of at least 25 mVal/100 g is used as layer silicate.

Activation preferably occurs by uniform spraying of the powdered, predried starting mineral (residual moisture content about 5 to 10%, preferably about 6 to 8%) or by intensive kneading of the essentially freshly mined starting mineral with the (mineral) acid. It was found that particularly advantageous and efficient adsorbents are obtained by this type of activation, especially by spraying with acid. In principle, however, acid activation can also be run so that the activation acid is added to a suspension of layer silicate. In this case, however, the water must be evaporated after acid activation so that energy demands of the process are increased. In order to achieve good mixing of the activation acid with layer silicate, on the one hand, and to keep the energy demands during evaporation water as low as possible, on the other, a suspension with the highest possible solids content is preferably used which can still be readily agitated.

The acid-activated layer of silicate is preferably dried before use and optionally subjected to further size reduction.

According to one variant, the layer silicate activated with acid is calcined before use, for example at a temperature of about 200 to 400° C. and optionally ground.

All strong acids can be used in general for acid activation, especially sulfuric acid, phosphoric acid, hydrochloric acid, formic acid and citric acid.

A mineral acid, like sulfuric acid, hydrochloric acid, nitric acid or phosphoric acid is preferably used. Sulfuric acid is particularly preferred, since this does not evaporate during acid activation so that activation can be run with a limited amount of acid. In addition, phosphoric acid and hydrochloric acid, especially in mixture with sulfuric acid, are also preferred.

The physical characteristics used to characterize the products according to the invention are determined as follows:

1. Ion exchange capacity (IEC)

The layer silicate being investigated is dried over a period of 2 hours at 150° C. The dried material is then made to react with a large excess of aqueous NH₄Cl solution for 1 hour under reflux. After a standing time of 16 hours at room temperature, it is filtered whereupon the filter cake is washed, dried and ground and the NH₄ content in the layer silicate determined according to Kjeldahl.

2. pH value of the starting material

10 g of a dried layer silicate is suspended during agitation in 100 mL distilled water for 30 minutes. After settling of the layer silicate, the pH value of the overlying solution is determined by means of a pH electrode.

3. Pore volume

The pore volume is determined according to the CCl₄ method (H. A. Benesi, R. V. Bonnar, C. F. Lee, Anal. Chem. 27 (1955), page 1963.

To determine pore volume for different pore diameter ranges, defined partial CCl₄ vapor pressures are adjusted by mixing the CCl₄ with paraffin.

4. Specific surface

This is determined according to the BET method (one-point method with nitrogen according to DIN 66131).

It was surprisingly found that the mycotoxin adsorbents activated with relatively small amounts of acid can adsorb both toxins adsorbable on acid surfaces, like fumonisin, and also toxins, especially aflatoxins, very quickly and stably.

The mycotoxin adsorbents used according to the invention are particularly suited for adsorption of mycotoxins from the group of aflatoxin, ochratoxin, fumonisin, zearalenone, deoxynivalenol and T2 toxin. Simultaneous effective adsorption of aflatoxin and non-aflatoxins, especially fumonisins and toxins from the trichothecene group (deoxynivalenol, T2 toxin and HT-2 toxin) is particularly advantageous.

It is assumed, without the invention being restricted to this theoretical action mechanism, that by activation with relatively limited amounts of acids, different surfaces are produced on the layer of silicate particles that offer optimal adsorption conditions for different mycotoxins. In comparison with conventional, i.e., layer silicates activated with larger amounts of acids and their mixtures, an increased adsorption capacity both for aflatoxins and non-aflatoxins was unexpectedly found with the nonactivated layer silicates. It is assumed that during adsorption of mycotoxins, surfaces of the layer silicate particles occupied with different intensity with acid cooperate so that a synergistic effect is produced. It is also assumed without restricting the invention to this that at least individual particles of the activated layer silicate according to the invention have a gradient with reference to acid occupation, in which the polyvalent cations situated close to the particle surface are primarily exchanged with H⁺. In the particle interior, however, sufficient polyvalent cations are still found, like Ca²⁺ and Al³⁺, in order to also permit efficient adsorption of other mycotoxins, like aflatoxins, by chelate formation. In contrast to this, this appears to be no longer attainable during unduly strong acid activation of the layer silicate because of the unduly sharp change in structure and composition of the crystal lattice.

According to a particularly preferred variant, layer silicates primarily activated on the particle surface with acid are used. The layer silicate is activated so that the polyvalent cations close to the surface of the layer silicate particles, but not those in the core region of the particles, are primarily exchanged. It was found that a high degree of surface acidity is achieved by the limited amounts of acids whereas during addition of higher amounts of acid, the surface acidity no longer increases significantly but the total acidity, measures as acidity of the clay suspension.

According to another aspect, the layer silicate has an ion exchange capacity (IEC) of at least 25 mval/g, preferably at least 5 mval/g, especially at least 45 mval/g.

It is assumed, without restricting the invention to this theoretical mechanism, that the IEC still (largely) present even after acid activation also contributes to efficient adsorption of mycotoxins. Different toxins are then protonated on the acid surface of the adsorbents used according to the invention and fixed as positively charged compounds on the available ion exchange sites.

According to another aspect, mycotoxin contamination involves two or more mycotoxins, especially aflatoxin(s) and at least one additional mycotoxin, like fumonisin, ochratoxin, deoxynivalenol and/or T2 toxin, especially fumonisin.

According to a preferred variant, referred to the amount of mycotoxin-contaminated material, at least 0.01 wt %, preferably at least 0.05 wt %, especially at least 0.1 wt % of acid activated layer silicate is used.

According to another advantageous variant, in addition to the layer silicate, an additional mycotoxin adsorbent is used. In principle, any mycotoxin adsorbent in the prior art can be used. An organophilic layer silicate and/or a layer silicate not activated with acid, especially bentonite, is preferably involved.

According to a particularly preferred variant, the acid-activated layer silicate and the additional mycotoxin adsorbents (adsorbent) are present in a mixture.

According to another aspect, the invention concerns a mycotoxin adsorbent containing a mixture of an acid-activated layer silicate according to one of the claims 1 to 6 and at least one additional mycotoxin adsorbent chosen from the group of layer silicates not activated with acid and organophilic layer silicates. Additional, generally organic components can be admixed with this mineral mixture, which according to the prior art are capable of improving utilization of the contaminated feed, stabilizing the health of the animals, especially their immune system or positively influencing metabolic processes. These include: vitamins, enzymes, plant components or extracts, as well as other substances known under the name probiotics.

According to another aspect, the invention concerns a feed preparation containing a mycotoxin-contaminated animal feed and an acid-activated layer silicate as just described, preferably in an amount of at least 0.01 wt %, preferably at least 0.05 wt %, especially at least 0.1 wt % of the contaminated animal feed.

Finally, the invention concerns, according to an additional aspect, a method for better usability or improvement of compatibility for animals and man of a mycotoxin-contaminated feed or food. A layer silicate activated with acid, as just described, is added to the feed before or simultaneously with absorption by an animal. According to this method of the invention, an improved weight increase can be achieved during feeding of acid-activated layer silicate with the mycotoxin-contaminated feed or food.

The invention is now explained in nonrestrictive fashion by means of the following examples:

### EXAMPLES

### Example 1

1. Acid activation of a Ca-bentonite

500 g (air dried) South African bentonite (natural Ca-bentonite) with a water content of 38 wt %, a BET surface of 63.4 m²/g, an IEC of 80.0 mval/100 g, pH value of 7.9 and a total pore volume (0-80 nm) of 0.120 mL/g was kneaded in a Werner-Pfleiderer mixer with addition of 150 mL distilled water and 19.38 g 96% H₂SO₄ for 10 minutes at about 30° C. The material was dried at 120° C. (to a residual moisture content of 9.5%) and ground on a Retsch impact mill (mesh width 0.12 mm). The ground product had the following characteristics:

BET surface: 58.5 m²/g

Pore volume: 0.10 mL/g

Sieve residue 63 μm: 28%

IEC: 77 mval/100 g

The material according to the invention so obtained is referred to below as C.

As comparison A, the crude bentonite not activated with acid but otherwise processed accordingly was used.

As comparison B, the crude bentonite, as activated above, but in which 88.4 g 96% H₂SO₄was used and kneaded for 10 minutes at about 30° C. was employed. Grinding and screening occurred as mentioned above.

As comparison E, a bentonite, Tonsil Optimum (Süd-Chemie AG) activated in a slurry with acid in the conventional manner was used.

2. Acid activation of halloysite

500 g (air dried) of Mexican halloysite ground on a Retsch impact mill (mesh width 0.12 mm) and predried to about 9% residual moisture content was sprayed on a granulation plate over 15 minutes uniformly with 3.5% (referred to air-dried mineral) in 96% sulfuric acid.

The product so produced had the following characteristics:

BET surface: 134 m²/g

Pore volume: 0.31 m²/g

IEC: 53 mval/100 g

The material so obtained according to the invention is referred to below as D.

3. Checking of adsorption efficiency

A) Adsorption

To conduct the adsorption experiments, aqueous solutions, each with 2000 ppb of the toxins aflatoxin B1 and fumonisin B1 were prepared. The solutions were set at pH 4.5 with citrate buffer.

0.25 g of the adsorbents/products described above were suspended in 50 mL of the solutions and agitated over 2 hours at a temperature of 37° C. The suspensions were then centrifuged for 5 minutes at 1500 rpm and the clear supernatant investigated by HPLC analysis for residual content of unadsorbed mycotoxin. The difference between the introduced amount of toxin and the amount of toxin still remaining in the solution after the described adsorption phase corresponds to the adsorbed amount and is stated in % of introduced amount of toxin.

B) Desorption

In a subsequent experiment, the possible desorption of the toxins adsorbed in the first step was investigated. For this purpose, the solids obtained after centrifuging of the suspension described in A were resuspended in 50 mL distilled water and set at pH 7. The now pH-neutral suspension was again agitated for 2 hours at 37° C. and then centrifuged. The amount of desorbed toxin was determined in the clear supernatant by HPLC analysis. The amount of toxin found in the solution was referred to the amount originally used in the adsorption experiment and stated in % of desorbed toxin.

The values stated in the tables for effective adsorption correspond to the difference from % of adsorbed amount minus % desorbed amount.

HPLC determination occurred under the following conditions:

| | Aflatoxin B1 | Fumonisin R1 |
|---|---|---|
| Column: | Spherisorb ODS-2 125 × | Lichrosphere 5 RP-18 |
| | 4 mm | 250 × 4 mm |
| Mobile | 600 mL 1 mmol NaCl | 385 mL methanol/115 mL |
| solvent: | solution/200 mL acetonitrile/ | 0.1 M NaH₂PO₄ with |
| | 200 mL methanol | H₃PO₄ set at pH 3.35 |
| Flow rate: | 1.5 mL/min | 1 mL/min |
| Detector: | Fluorescence | Fluorescence |
| Wavelength: | EX 365 nm/EM 455 nm | EX 335 nm/EM 440 nm |
| Furnace | 35° C. | 40° C. |
| temperature: | | |
| Injection | 100 μL | 100 μL |
| volume: | | |

The effective adsorption value so obtained (% adsorption-% desorption) are summarized in Table 1.

Some properties of the employed materials are summarized in Table 2.

TABLE 1

| | | Effective adsorption (%) | | | | |
|---|---|---|---|---|---|---|
| Mycotoxin | | A | B | C | D | E |
| Aflatoxin B1 | pH 7 | 91.3 | 78.4 | 95.8 | 97.3 | 56.5 |
| Aflatoxin B1 | pH 4 | 96.5 | 80.6 | 97.5 | 98.6 | 64.0 |
| Fumonisin | pH 7 | 9.2 | 92.7 | 92.0 | 93.1 | 92.4 |
| Fumonisin | pH 4 | 13.4 | 94.9 | 94.7 | 95.8 | 94.1 |

TABLE 2

| | | BET | Pore | IEC |
|---|---|---|---|---|
| | | surface | volume | mval/ |
| Materials | | m²/g | mL/g | 100 g |
| A | Crude bentonite South Africa | 63 | 0.12 | 80 |
| B | A + 14% H₂SO₄ kneaded | 45 | 0.08 | 52 |
| C | A + 3.5% kneaded | 58 | 0.1 | 77 |
| D | Halloysite Mexico + 3.5% H₂SO₄ sprayed | 134 | 0.22 | 53 |
| | on (crude material Halloysite Mexico) | | | |
| E | Tonsil Optimum 210 FF (conventional | 200 | 0.29 | 16 |
| | slurry activation) | | | |

It is apparent from Table 1 that the mycotoxin adsorbents C and D according to the invention excellently adsorbed both the aflatoxin and the non-aflatoxin fumonisin. The effect of adsorption rate of the mycotoxin adsorbent C according to the invention was surprisingly even above that of the natural bentonite A (not activated with acid) and the rate for fumonisin above that of bentonite B activated with larger amounts of acid. This was all the more so unexpected, since according to the prior art aflatoxins are adsorbed particularly well on layer silicates not activated with acid, and fumonisin, for example, is adsorbed particularly well on conventional (uniformly) acid-activated layer silicates.

1. Use of a layer silicate activated with acid for adsorption of mycotoxins, characterized by the fact that the layer silicate is activated with less than 10 wt % referred to air-dried layer silicate, of an acid at a temperature below 80° C.

2. Use according to claim 1, characterized by the fact that the layer silicate is activated with about 0.5 to 8 wt %, especially about 1 to 6 wt %, and particularly about 1.5 to 4 wt % acid.

3. Use according to claim 1 or 2, characterized by the fact that activation occurs by spraying with layer silicate with an acid or acid solution of kneading in of the acid.

4. Use according to one of the preceding claims, characterized by the fact that acid activation is run over less than 2 hours, especially less than 1 hour, at a temperature between room temperature (20° C.) and about 60° C.

5. Use according to one of the preceding claims, characterized by the fact that the layer silicate activated with acid is used in feeds.

6. Use according to one of the preceding claims, characterized by the fact that the layer silicate is chosen from the smectite group, the attapulgite/palygorskite group, the group of vermiculites, illites, the serpentine-kaolin group, the talc-pyrophyllite group and/or the mica-like layer silicates.

7. Use according to one of the preceding claims, characterized by the fact that the layer silicate includes a montmorillonite clay, especially bentonite, as well as natural mixtures of attapulgite and halloysite.

8. Use according to one of the preceding claims, characterized by the fact that the layer silicate has an ion exchange potassium (IEC) of at least 25 mval/g.

9. Use according to one of the preceding claims, characterized by the fact that the acid-activated layer silicate is calcined at a temperature of about 200 to 400° C. before use and optionally ground.

10. Use according to one of the preceding claims, characterized by the fact that at least one additional mycotoxin adsorbent is used in addition to the acid-activated layer silicate according to the preceding claims.

11. Use according to claim 10, characterized by the fact that the additional mycotoxin adsorbents includes the layer of silicate, especially a montmorillonite clay, preferably a bentonite.

12. Use according to claim 11, characterized by the fact that the layer silicate is a layer silicate not activated with acid, especially Ca or Na bentonite and/or an organophilic layer silicate, especially an organophilic bentonite.

13. Use according to one of the preceding claims, characterized by the fact that the employed mycotoxin adsorbents are present in a mixture.

14. Use according to one of the preceding claims, characterized by the fact that the mycotoxin being adsorbed include one or more toxins chosen from the group of aflatoxins, citrinin, cyclopiazonic acid, ochratoxin, patulin, representatives of the trichothecenes like nivalenol, deoxynivalenol, T2 toxin, HT-2 toxin, fumonisins, zearalenone and ergot alkaloids.

15. Use according to one of the preceding claims, characterized by the fact that the mycotoxin being adsorbed include additional toxins, like ochratoxin, fumonisin, zearalenone, deoxynivalenol and/or T2 or T2-like toxins, in addition to aflatoxin(s).

16. Use according to one of the preceding claims, characterized by the fact that, referred to the amount of mycotoxin-contaminated material, at least 0.01 wt %, preferably at least 0.05 wt %, especially at least 0.1 wt % of the acid-activated layer silicate is used.

17. Mycotoxin adsorbent containing a mixture of an acid-activated layer silicate according to one of the claims 1 to 9 and at least one additional mycotoxin adsorbent chosen from the group of layer silicates not activated with acid and organophilic layer silicates.

18. Mycotoxin adsorbent according to claim 17, additionally containing organic compounds suitable for adsorption of mycotoxins, like ion exchanges or activated carbons, as well as optionally organic compound that permit improved utilization of the mycotoxin-containing feeds and stabilized metabolic processes in the animal, like vitamins, trace nutrients and probiotics.

19. Feed preparation, containing a mycotoxin-contaminated animal feed and an acid-activated layer silicate according to one of the claims 1 to 9, preferably in an amount according to claim 16.

20. Method for improvement of usability of a mycotoxin-contaminated feed, characterized by the fact that an acid-activated layer silicate according to one of the claims 1 to 9 is added to the feed before or simultaneously with absorption by an animal.
